(19) United States
(12) Plant Patent Application Publication
(10) Pub. No.: US 2003/0009797 P1
(43) Pub. Date: Jan. 9, 2003

(54) Peach variety named "Southern star"

(76) Inventors: Michael T. Malone, Havelock North (NZ); Paul G. Glucina, Auckland (NZ)

Correspondence Address: GREENLEE WINNER AND SULLIVAN P C, 5370 MANHATTAN CIRCLE, SUITE 201, BOULDER, CO, 80303, US

(21) Appl. No.: 10/170,218

(22) Filed: Jun. 10, 2002

(30) Foreign Application Priority Data
Jun. 11, 2001 (NZ) SFM 104

(51) Int. Cl. A01H 5/00

(52) U.S. Cl. PLT/195

(57) ABSTRACT

A new and distinct variety of peach tree (Prunus persica), named ‘Southern Star’, is described. The new variety is a selection derived from the open pollinated seed of ‘Yumyeong’ (not patented) and is distinguishable from the parent variety by ripening 1 week earlier than ‘Yumyeong’ and having sweeter, more aromatic fruit. Its novelty consists of a unique combination of features that include late flowering, heavy and regular cropping, large, very firm, white flesh, crisp-textured, clingstone fruit with a red overcolour. Fruit have a sweet, sub-acid flavour.

### CROSS REFERENCE TO RELATED APPLICATIONS

This application claims benefit of New Zealand PVR Application SFM 104, filed Jun. 11, 2001.

### BACKGROUND OF THE INVENTION

The present new and distinct variety originated from an open pollinated population of seedlings derived from the variety ‘Yumyeong’ (not patented) planted in 1990 at the HortResearch experimental orchard in Goddard Lane, Havelock North, New Zealand. A large group of open-pollinated seedlings on their own roots were placed under careful observation. One seedling representing the present variety was selected and asexually propagated for further evaluation. The selection was further propagated by asexual reproduction for the first time in 1995.

The new variety was budded onto ‘Golden Queen’ peach seedling rootstock and shows the unique combination of characteristics and distinctive colour are true to the original seedling tree and are transmitted through succeeding asexual generations.

The variety is characterised by late flowering, vigorous spreading growth; regular and heavy cropping of large, very firm,white-fleshed crisp-textured fruit which are clingstone with mild and sweet sub-acid flavor and fruit aroma. The fruit is further characterised by pink over-colour and very good handling qualities, and ripening 1 week before the parent ‘Yumyeong’.

### SUMMARY OF THE INVENTION

The described variety of peach tree is of a medium to large size, vigorous, with a spreading growth habit. Trees of the variety bear regular and productive crops of medium to large white flesh, clingstone fruit with a sweet sub-acid taste and very firm crisp flesh. Fruit has very good flavour and eating quality. The firm flesh exhibits good storage qualities and the skin has red colour over a cream background. The fruit ripens mid season approximately 1 week before the ‘Yumyeong’ parent variety.

### BRIEF DESCRIPTION OF THE DRAWINGS

FIG. 1 shows the exterior view of the fruit on the tree at harvest.

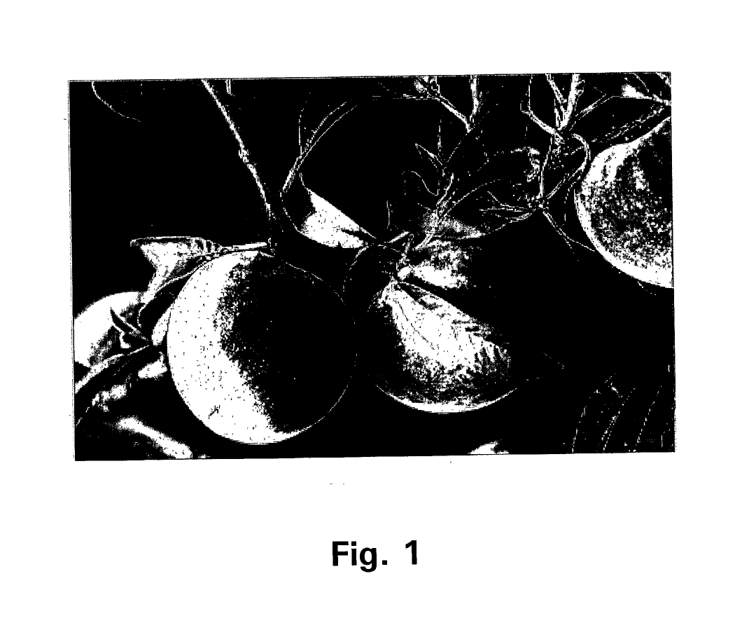

FIG. 2 shows external and internal views of the fruit. The photograph was taken after 2 weeks air storage of fruit harvested at eating maturity.

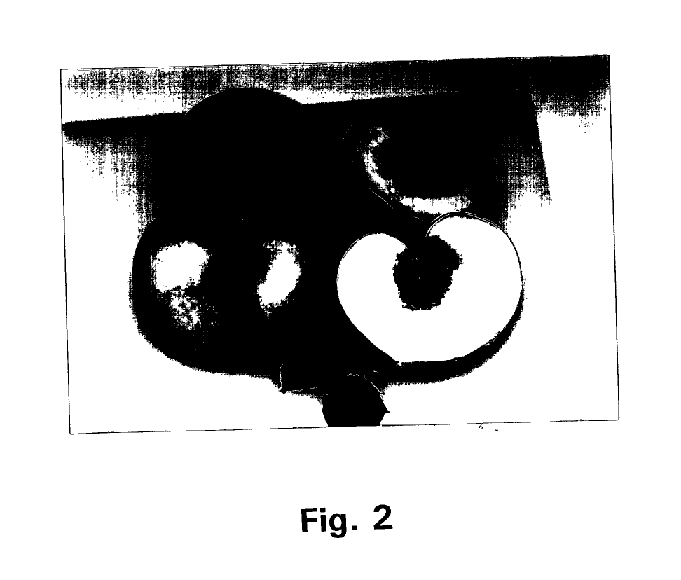

### DETAILED DESCRIPTION OF THE NEW VARIETY

The following is a detailed description of the new variety with colour terminology in accordance with The Royal Horticultural Society Colour Chart (RHSCC) 2000 edition. The specimens described were grown in Havelock North and Clyde, Central Otago, New Zealand. The observations were made in the 2001 season on trees that were four years old at the time.

The present new variety of peach tree, the flowers, foliage and fruit herein described may vary slightly in detail due to climate, soil conditions and cultural practices under which the variety may be grown. Unless otherwise stated the present description is that of the variety grown under the environmental conditions prevailing near Havelock North, Hawke's Bay New Zealand.

Tree:

Size.—Large.

Vigour.—Vigorous

Form.—Pruned to central leader, with a spreading growing habit.

Trunk:

Size.—Medium

Texture.—Medium rough

Colour.—Grey/brown (RHS Fan 4 197A)

Branch:

Size.—Medium

Texture.—Smooth-rough

Lenticels.—Medium number, large elongated shape, varies with age of growth

Leaves:

Size.—Medium to large, average width 5.5 cm and average length 19.0 cm Form. Pointed. Shape of tip acuminate, angle of tip is narrow acute, shape of the base is attenuate, incision of margin is crenate.

Thickness.—Medium

Surface.—Smooth

Petiole.—Medium length, average length 1.5 cm, medium thickness,

Glands.—Reniform, elongated, predominant number of nectaries on fully developed leaf more than two, average 4.

Colour.—Upper surface dark green (RHS Fan 3141A), lower surface lighter green (RHS Fan 3 146B)

Flower buds:

Size.—Medium to large

Length.—Medium

Form.—Plump

Flowers:

Size.—Medium to large

Pollen.—Present

Colour.—Medium pink.

Petal shape.—Round

Pubescence of ovary.—Present

Blooming period.—The trees flowered in mid-late September. Date of 5% bloom approximately 20 September, full bloom 30 September, 5% petal fall 7 October 2000 in Havelock North. Full bloom 3 October in Clyde.

Fruit:

Harvest maturity: Fruit ripened for eating mid season. Harvest usually commences about January 5-10 in Hawke's Bay and February 7-19 in Clyde, New Zealand.

Date of first picking: Feb. 7 2001 in Clyde, New Zealand. Mid-late season.

Date first pick: January 14 in Havelock North, New Zealand.

At maturity for consumption, ripe:

Size.—Medium, average weight 170 grams. Diameter across suture line average 173 mm average length 164 mm.

Shape.—In ventral view, round oblate, symmetrical

Shape of pistil end.—Rounded, slightly depressed.

Suture.—Shallow, extends from base to apex

Cavity.—Rounded, medium to deep, average depth 9 mm, average width 28 mm

Flesh:

Ripen.—Evenly

Texture.—Firm non-melting

Fibres.—Non fibrous

Aroma.—Slight

Flavour.—Good, low acid, mild.

Soluble solids.—Average 10-12 Brix

Juice.—Moderate juicy

Colour.—White (RHS Fan 4 155D)

Anthocyanin.—Low

Stem:

Size.—Medium, average 0.5 cm

Colour.—Bright green (RHS Fan 3 150C)

Skin:

Thickness.—Medium

Texture.—Medium to strong adherence of skin to flesh Density of pubescence, medium Tendency to crack, none

Colour.—Red (RHS Fan 1 46A), blending to a pink (RHS Fan 1 48A) blush pattern of over colour. Cream ground colour of skin (RHS Fan 1 11D)

Stone:

Type.—Freestone

Size.—Medium, average stone length 32 mm, average stone width 26 mm, average stone thickness 17.5 mm, average stone weight 6.5 grams.

Shape.—In lateral view, round/elliptic

Surface.—Relief of surface, pits and grooves, pitted towards the base

Tendency to split.—None

Colour.—Brown (RHS Fan 4 165A)

Storage: Trials indicate 2-3 weeks air storage without internal breakdown of flesh.

1. A new and distinct peach tree substantially as herein described and illustrated characterized by late flowering, vigorous spreading growth; regular and heavy cropping of large, very firm, white-fleshed crisp-textured fruit which are clingstone with mild and sweet sub-acid flavor and fruit aroma, the fruit being further characterised by pink over-colour and very good handling qualities.
